(19) United States
(12) Patent Application Publication
(10) Pub. No.: US 2012/0020925 A1
(43) Pub. Date: Jan. 26, 2012

(54) Therapeutic Use of Specialized Endothelial Progenitor Cells

(76) Inventors: Robert I. Grove, Avon, IN (US); Christine Smith-Steinhart, Indianapolis, IN (US); Paul A. Hyslop, Indianapolis, IN (US)

(21) Appl. No.: 13/146,137

(22) PCT Filed: Feb. 3, 2010
(86) PCT No.: PCT/US2010/022996

§ 371 Date: Jul. 25, 2011

Related U.S. Application Data
(60) Provisional application No. 61/149,872, filed on Feb. 4, 2009.

(51) Int. Cl. A61K 35/12; C12N 5/071; A61P 17/02; A61P 9/00; A61P 9/04; C12Q 1/68; A61P 9/10

(52) U.S. Cl. 424/93.3; 435/6.1; 435/325

(57) ABSTRACT

The invention relates to products, processes, and therapeutic methods for restoring blood flow to tissues at risk of becoming or being ischemic by inducing angiogenesis and/or vasculogenesis in tissues in need thereof by administering endothelial colony forming cells alone or in combination with other cell types and/or agents. In one aspect, the invention is useful for inducing angiogenesis and/or vasculogenesis in patients with ischemic disease including ischemic heart disease, and other ischemic vascular disorders.

This application claims priority from U.S. Provisional Application Ser. No. 61/149,872 the entire contents of which is hereby incorporated by reference.

### TECHNICAL HELD

The present invention relates generally to products and methods pertaining to the angiogenic and vasculogenic potential of highly proliferative endothelial progenitor cells, termed ECFCs, for various purposes including therapeutic use in mammals.

### BACKGROUND OF THE INVENTION

Ischemia is a medical condition characterized by insufficient oxygenation to any part of the body. As an example, peripheral vascular disease (PAD) is an ischemic condition caused by obstruction of arteries and blood flow to the leg. PAD is particularly prevalent among the elderly affecting approximately 10.3 million people in the U.S. Many patients with PAD experience debilitating leg pain with exertion. Despite aggressive treatment with currently-available therapies, 25% of patients with PAD require partial amputation of one leg within a year of diagnosis. This represents over 100,000 people annually in the U.S. By five years, only half of these patients will still have both legs intact, and nearly 20% of the initial patients will have died from this or a related ischemic disease.

Ischemia can also lead to heart disease. About 1.2 million Americans suffer a heart attack every year, with approximately 40% resulting in death. Many heart attacks are traceable to coronary artery disease (CAD) which results in partial or complete obstruction of cardiac blood vessels. While roughly 60% of patients survive a heart attack, frequently there is permanent heart muscle damage. Even in the best-case scenarios, cardiologists are only able to save about 60% of cardiac muscle after a heart attack. According to MANES the total prevalence of all forms of coronary heart diseases in the United States exceeded 13 million in 2003, at a staggering cost of $403 billion. (Heart Disease and Stroke Statistics—2008 Update: Circulation, 2008; 117 e25-e146). Growing public awareness of the early signs of heart disease, and improved medical techniques for accurate diagnosis, has enhanced the potential for interventional therapy to avert heart attacks. Improvements in minimally-invasive approaches to treat patients in early and late-stages of cardiac disease—including pharmacological, neovascularization, and/or cell-based treatments, are expected to reduce the frequency and severity of heart failure. A critical component in the success of these objectives is successfully addressing the problem of myocardial ischemia.

Chronic myocardial ischemia leads to a decrease in heart function, and eventually death of the ischemic heart tissue. It is believed that heart function could be salvaged and even restored to normal by stimulation of biological processes known as vasculogenesis and angiogenesis (Melero-Martin, J. M. et al., In Meth, Enzymol., 445, 303-329 (2008) Angiogenesis denotes the formation of new blood vessels from pre-existing ones, whereas vasculogenesis refers to the formation of new blood vessels de now. Vasculogenesis occurs mainly during embryologic development, and is associated with endothelial colony forming cell (ECFC) migration and differentiation in response to local factors such as growth factors and extracellular matrix to form new blood vessels. Newly formed vascular trees are then pruned and extended through angiogenic processes.

Recently, it was discovered that vasculogenesis also occurs in the adult organism. Melero-Martin, J. M. et al., Circ. Res., 103, 194-202 (2008). Circulating ECFCs contribute, albeit to varying degrees, to neovascularization, such as occurs during tumor growth, or in revascularization following trauma.

Means for inducing vasculogenesis and angiogenesis may prove to be important in treating and/or preventing myocardial ischemia and other ischemic-related conditions. Areas of the myocardium following an ischemic episode become necrotic, the tissue dies and fibrotic scar tissue forms. Functional remodeling of fibrotic tissue may occur if a de novo blood supply can be established as a first step to providing nutrients and gas exchange capabilities to the necrotic tissue. Establishment of a de nom blood vessel network in an ischemic area that inosculates with existing surrounding vasculature of functional tissue is characteristic of the regenerative process. Once this process has occurred, local angiogenic processes provide an appropriate degree of vessel density as the tissue remodels and regains functionality.

Surgical interventions can be highly successful in treating various ischemias including myocardial ischemia. For example, coronary artery bypass surgery or angioplasty can, in most cases, reduce the symptoms of myocardial ischemia. However, alternatives to surgical intervention are desirable for a variety of reasons including the potential to reduce medical costs, discomfort, and recovery time for patients. In this regard, pharmacological interventions are of increasing interest as a possible means to stimulate the body's ability to generate new blood vessels. For example, administering one or more growth factors such as FGF-1, FGF-2, FGF-5, PDGF-1, PDGF-2, VEGF, and IGF has received considerable research interest. Other potential therapies include cell-based treatments which are of increasing interest for the treatment and/or prevention of ischemic disorders.

Cell-based treatments present new options for treating ischemic disorders involving the delivery of progenitor cells or stem cells to the damaged site in order to facilitate vascular restoration. Several studies have shown that bone marrow-derived cells, or cells circulating in peripheral blood contribute to neoangiogenesis in wound healing, limb ischemia, and post-myocardial infarction (See, e.g., Rafii. S, and Lyden, D., Nat. Med. 6, 702-712, 2003). While technical advances in cell-based treatments appear promising, adequate revascularization of smaller peripheral vessels remains a challenge. Furthermore, if revascularization does occur, it is often too slow to completely restore organ function and prevent further damage to the tissue. Additionally, the use of whole populations of bone marrow mononuclear cells which contain different organ-specific stem, progenitor and hematopoietic cells may be accompanied by new toxicity concerns (e.g., Arora et al., Biol. Blood & Marrow Transplant 13 145, 2007).

There remains a need for effective cell-based methods to enhance angiogenesis and/or vascular growth and function in mammalian tissues and for effective means to treat diseases in humans including ischemic disorders. With improvements in minimally-invasive approaches such as direct injection of angiogenic and arteriogenic therapeutics to treat patients with early and late-stage disease through neovascularization treatments, the frequency and severity of cardiovascular and other ischemia-related diseases is expected to be significantly reduced. The present invention provides compositions, products and methods to improve blood flow to mammalian tissues including human tissues by administration of a cell-based composition.

### SUMMARY OF THE INVENTION

It is an object of the present invention to provide highly proliferative endothelial colony forming cells for industrial, research, and/or therapeutic applications including enhancing blood flow to mammalian tissue in need thereof.

It is another object of the present invention to provide cell-based methods to prevent, alleviate, or treat diseases and conditions in mammals including humans that arise from or are associated with potential or actual ischemia and damage relating to reduced or retarded blood flow (i.e. lack of oxygen supply), including, but not limited to diseases and conditions of the myocardium, congenital heart deficit, heart valve disease, arrhythmia, left ventricular dilation, emboli, heart failure, coronary artery disease (CAD), angina, subendocardial fibrosis, left or right ventricular hypertrophy, myocarditis, myocardial infarction (MI), congestive heart failure (CHF), stroke, peripheral artery disease (PAD), wound healing and/or other ischemic diseases.

It is another object of the invention to provide a method to induce angiogenesis and/or vasculogenesis in mammalian tissues including but not limited to ischemic or potentially ischemic tissues to restore and/or enhance blood flow by generating or regenerating, blood vessels for use in clinical applications including but not limited to myocardial infarction, peripheral vascular disease, coronary artery disease, would healing, stroke, renal artery disease, diabetic ulcer healing and congestive heart failure.

It is another object of the present invention to provide a composition comprising highly proliferative endothelial colony forming cells comprising karyotypically normal ECFCs for therapeutic purposes including treating an ischemic disorder in a patient in need of such treatment.

It is another object of the present invention to provide a kit that comprises ECFCs including cryopreserved ECFCs, and optionally one or more other agents including cryopreserved or freshly isolated helper cells such as adipose stromal cells (ASCs), pharmaceutical carriers, excipients, buffers, proteins, peptides, matrix materials, and/or other agents for use in treating ischemic disorders.

Still another object of the invention relates to methods and/or processes for producing, improving and/or enhancing the angiogenic potential of ECFCs.

These and other objects of the invention are evidenced by the summary of the invention, the description of the preferred embodiments and the claims.

In one embodiment, the invention relates to a composition of matter comprising karyotypically normal ECFCs.

In another embodiment, the present invention relates to a product-by-process comprising karyotypically normal ECFCs.

In another embodiment, the present invention relates to a method for treating a patient with an ischemic disease including myocardial infarction (MI), coronary artery disease (CAD), congestive heart failure (CHF), stroke, peripheral artery disease and other ischemic disease including myocardial ischemia, chronic myocardial ischemia and acute myocardial ischemia comprising administering an effective amount of ECFCs to stimulate angiogenesis and/or vasculogenesis.

In another embodiment, the present invention relates to a method for increasing blood flow or perfusion to a site in a patient in need thereof, including a site of ischemic injury, comprising administering ECFCs.

In another embodiment, the present invention relates to as method to reverse, limit or prevent ischemic damage and/or tissue death in a patient in need thereof by inducing angiogenesis and/or vasculogenesis comprising administering an effective amount of ECFCs.

In another embodiment, the present invention relates to a method to reverse, limit or prevent vascular damage associated with an ischemic disease or condition comprising administering an effective amount of ECFCs.

In another embodiment, the present invention relates to a method to reverse, limit or prevent cardiac cell apoptosis comprising administering ECFCs.

In another embodiment, the present invention relates to a method to reverse, limit or prevent the effects of ischemic disease comprising administering ECFCs in combination with one or more helper cell types, or other source of smooth muscle cells and/or pericytes, preferably also including a suitable matrix material to a patient in need thereof.

In another embodiment, the present invention relates to a method for inhibiting or reducing fibrosis associated with ischemia including but not limited to myocardial ischemia by administering ECFCs alone or in combination with one or more suitable helper cell types and a matrix material to a patient in need thereof.

In another embodiment, the invention relates to administering ECFCs alone or in combination with helper cells including but not limited to those selected from the group consisting of adipose stromal cells, bone marrow mononuclear cells, and endometrial mesenchymal cells, or Wharton's jelly mesenchymal cells to enhance blood flow and reverse, limit or prevent ischemic diseases including myocardial infarction (MI), congestive heart failure (CHF), stroke, peripheral artery disease (PAD), and other ischemic disease.

In another embodiment the present invention relates to administration of ECFCs in combination with one or more other pharmacological agents including but not limited to, β-blockers, diuretics, Ca-channel blockers and ACE inhibitors to treat or prevent a disease including myocardial infarction (MI), congestive heart failure (CHF), stroke, peripheral artery disease (PAD), and ischemic disease or condition.

In another embodiment, the present invention relates to administering to a patient in need thereof a composition comprising ECFCs alone or in combination with helper cells and optionally containing a matrix material in conjunction with an adjunct procedure including but not limited to prosthetic device(s) including stems and vascular prostheses.

In another embodiment, the present invention relates to a method for treating myocardial infarction (MI), congestive heart failure (CHF), stroke, peripheral artery disease (PAD), coronary artery disease (CAD), and ischemia comprising administering ECFCs in combination with one or more other biologically active agents including proteins, peptides, and growth factors.

In yet another embodiment the present invention relates to a pharmaceutical composition comprising a therapeutically effective amount of ECFCs alone or in combination with one or more other agents including helper cells and/or other pharmacological agents including but not limited to growth factors, β-blockers, diuretics, Ca-channel blockers, ACE inhibitors, and optionally also including a suitable matrix material.

This Summary is provided merely to introduce certain concepts and unless otherwise indicated not to identify any key or essential features of the claimed subject matter.

### BRIEF DESCRIPTION OF THE DRAWINGS

FIG. 1A (top panel). Left ventricular end diastolic diameter (LVEDD) at 4 weeks after induction of cardiac ischemia in nude rats showing control and ECFC-treated animals. ECFC-treated animals show a significant improvement in reduction in diameter compared with negative control, closely approximating normal diameter.

FIG. 1B (bottom panel). Percent Left Ventricular Shortening at 4 weeks after induction of cardiac ischemia in nude rats showing normal, control, and ECFC-treated animals. ECFC-treated animals show an approximate 50% improvement over control animals.

FIG. 2. Capillary density in stained sections of infarcted rat heart at two and four weeks after induction in control (left panels, top and bottom) and ECFC-treated animals (right panels, top and bottom).

FIG. 3. Capillary density (per mm²) in control and ECFC-treated infarcted rat heart.

FIG. 4. Histological section of normal human colon. Nuclei stained brown using human anti-nuclear matrix protein. CD31 stained red using human anti-CD31.

FIG. 5. Histological section of normal rat myocardium injected with a mixture of ECFCs, ASC, and Extracel™ matrix material.

FIG. 6. Histological section of experimentally induced ischemic rat myocardium injected with mixture of ECFCs, ASCs and Extracel™ matrix material. Arrows labeled “B” identify human nuclei in cells that have integrated into rat blood vessel linings.

FIG. 7. Histological section of experimentally induced ischemic at myocardium taken from a portion of heart adjacent to site of injection.

### DETAILED DESCRIPTION

As used herein, the term “HPP-EPC” refers to a subclass of endothelial progenitor cells isolated from cord blood haying high proliferative potential as described in WO/2005/078073.

As used herein, the term “ECFCs” refers to endothelial colony forming cells isolated from placenta cord, cord blood, or other suitable source. In a preferred embodiment, ECFCs exhibit a normal karyotype. Karyotypically normal ECFCs may be obtained by expansion HPP-EPC according to methods described herein from clones having a normal karyotype. A commercially available source of preferred ECFCs are ECFCs® isolated from cord blood, produced by EndGenitor Technologies, Inc. Indianapolis, Ind.) and are available for research use from Dynacell Life Sciences, LLC 19 Hendricks St. Ambler; PA 19002 and Lonza Walkersville, Inc., 8830 Biggs Ford. Road City, Walkersville. County, Md., 21793.

The terms “inosculate', anastomose”, “anastomosis”, and “anastomoses” refer to a biological process, or result thereof, in which a physical connection is made between tubular structures, including blood vessels.

As used herein, the term “therapeutically-effective” means providing a clinically relevant benefit to a patient, e.g. in treating myocardial ischemia as measured, for example, by increased cardiac perfusion, reduced angina, and/or neovascularization.

As used herein the term “ASC” refers to adipose tissue stromal cells including cells from a human or other mammalian sources.

The term “angiogenesis” as used herein means any physiological process involving the growth of new blood vessels from pre-existing vessels

The term “vasculogenesis” as used herein is the term used for spontaneous formation of new blood vessels from vascular cells

The term arteriogenesis refers to an increase in the diameter of existing arterial vessels.

The term “normal karyotype” or “substantially normal karyotype” or “karyotypically normal” as applied herein to a cell or population of cells means that the proper number of chromosomes are present and not noticeably altered. In a population of cells, greater than about 95%; more preferably greater than about 98%; still more preferably greater than about 99% of cells exhibit this characteristic as detected by any suitable means. In one embodiment, karyotypically normal ECFCs (i.e. ECFCs®) are produced by harvesting growing cells prior to confluence as described herein.

As used herein the term “matrix” or “matrix material” or “pharmaceutically acceptable matrix” or “extracellular matrix” means any suitable biocompatible polymer including synthetic or recombinantly engineered peptide or protein polymers. For example, such polymer matrices include but are not limited to matrix materials containing collagen and/or fibronectin, hydrogels containing a combination of native or modified hyaluronan, heparin, and gelatin, crosslinked by polyethylene glycol diacrylate, PEGDA. In some embodiments of the invention, a matrix material is admixed with cells prior to administration which is thought to concentrate cells at a particular location.

The term “co-administer” or “co-administration” refers to therapeutic administration to a subject including a human patient of more than a single agent either simultaneously or sequentially. For example, co-administration of two different cell types could involve a single administration of a dosage form having two different cell types in admixture, or one or more administrations to a patient of a dosage form having one cell type which precedes administration of a dosage having the other cell type.

The term “helper, or “helper cells” or “helper cell population” is used herein to mean any cell population, cell-based material, or composition of matter that when combined with ECFCs facilitates angiogenesis and/or vasculogenesis when administered to a mammal in need thereof. The “helper” or “helper cells” are believed to provide a suitable source of pericytes and/or smooth muscle cells that enhance angiogenesis and/or vasculogenesis when administered or co-administered in vivo to a mammal including a human. For example, a suitable helper includes but is not limited to adipose stromal cells and/or endometrial mesenchymal cells alone or in combination with one or more other helper cell type(s). The term “helper cells” is intended to broadly include any suitable source of smooth muscle cells and/or pericytes.

The term “ischemic disease” or “ischemia” as used herein refers to diseases and/or condition characterized by reduced oxygenation to any tissue in the body such as but not limited to, ischemic heart disease, transient ischemic attack (TIA), cardiac ischemia, stroke, reperfusion injury, bowel ischemia, intestinal ischemia, peripheral artery disease, critical limb ischemia, mesenteric ischemia, brain ischemia, leg ischemia, myocardial infarction, peripheral vascular disease, coronary artery disease, angina, wound healing, renal artery disease, diabetic ulcer healing, congestive heart failure, and hepatic ischemia. Ischemia can be caused by a number of conditions including but not limited to anemia, stroke and atherosclerosis. Multiple diseases result from ischemia including, for example, cerebrovascular ischemia, renal ischemia, pulmonary ischemia, limb ischemia, myocardial ischemia, and ischemic cardiomyopathy. The description that follows focuses on myocardial ischemia, but it should be understood that the method(s) and compositions described herein are also intended for treating and/or preventing ischemic diseases and conditions in tissues and organs other than heart.

As used herein with reference to administration of ECFCs and/or other cells to patients in need thereof, the terms “dosage”, “amount”, or “number,” are used essentially interchangeably to indicate the quantity of ECFCs, preferably ECFCs®, or other cells to be administered to achieve clinical benefit.

The methods and pharmaceutical compositions of the invention can be used to promote arterial vascular growth, for example in the treatment or prevention of conditions associated with ischemia. Such conditions include, but are not limited to, stroke or heart attack. In addition, the products, methods, and compositions of the invention can be used to accelerate wound healing, promote vascularization of surgically transplanted tissue, and enhance the healing of a surgically-created anastomosis.

The methods and compositions of the invention are effective for enhancing blood flow to biological tissues, including treating a patient suffering from, or at risk of suffering from, ischemic damage to an organ or tissue including but not limited to myocardial tissue. Reduced blood supply to a tissue can be caused by a vascular occlusion, resulting from, for example, arteriosclerosis, trauma, or surgical procedure. Determining whether a tissue is at risk of or has already been affected by ischemic damage due to vascular occlusion is readily ascertainable using any suitable technique known to the skilled artisan including a variety of imaging techniques (e.g., radiotracer methodologies, such as 99 mTc-sestamibi, x-ray, and MRI angiography) and physiological tests.

Induction of vascular growth in a tissue affected by or at risk of being affected by ischemia, using the methods and compositions of the invention, is expected to prevent, treat or reverse ischemia, regardless of its origin, in organs or tissues including, but not limited to, brain, heart, pancreas, or limbs.

Previous studies using human adult peripheral CD34 cells have shown improved heart function and increased blood vessel density in an immunodeficient rat model of myocardial ischemia. Kawamoto, A. et al. Circulation 2006 114 2163-9. Although the evidence supported an increase in angiogenesis induced by the CD34⁺ cells, the mechanism by which these cells improved function was not defined.

HPP-EPC are a subpopulation of endothelial progenitor cells described in WO 2005/078073, WO 2008/101100, US 2008/0025956, and M. C. Yoder et al., Blood, 109, 1801-1809 (2007), the entire contents of which are hereby incorporated by reference. Unlike other endothelial progenitors. HPP-EPC represent a highly proliferative subpopulation of endothelial progenitor cells (EPC) distinct from endothelial cell colony forming units (CFU-ECS) and from endothelial outgrowth cells (EOCs). HPP-EPC are obtained by ex vivo expansion and selection from, for example, human umbilical cord blood or adult peripheral blood or blood vessels, e.g. umbilical vein and human aortic vessels as described in WO 2005/078073. HPP-EPC express cat surface antigens that are characteristic of endothelial cells including CD31, CD105, CD 46, and CD144, but do not express antigens that are characteristic of hematopoietic cells, such as CD45 and CD 14. HPP-EPC are obtained by passaging cells at 90% to 100% confluence as described in WO 2005/078073.

### A. Isolation of Karyotypically Normal ECFCs

Passaging rapidly growing stern cells such as HPP-EPC can result in unstable karyotype cytogenetic abnormalities including the loss or gain of whole chromosomes, translocations, inversions, large-scale deletions and duplications. Many such abnormalities are known to cause or be associated with diseases in humans including Turner Syndrome, Kleinfelter Syndrome, Down's Syndrome, Cri du chat, and Angelman Syndrome, to name a few. Chromosomal abnormalities are also known to occur certain cancerous cells. For example, chronic myelogenous leukemia is associated with a chromosomal translocation resulting in the so-called Philadelphia chromosome.

To mitigate possible safety and/or efficacy concerns associated with administrating to patients stem or progenitor cells having chromosomal abnormalities it is desirable to produce and/or purify a population of ECFCs having a normal or substantially normal karyotype.

The phenomenon of chromosome instability has been observed by the present inventors in preparing HPP-EPC by published methods, that is by passaging cells at or near confluence, i.e. greater than 90% confluent, and this has led to a search for methods to prevent the incidence of unstable karyotype during preparation of ECFCs.

In one embodiment, the present invention relates to a population of clonally purified, high proliferative endothelial colony forming cells which have a substantially normal karyotype. ECFCs having a substantially normal karyotype can be produced from various tissue sources including cord blood by any suitable method including methods described herein. Methods pertaining to this aspect of the invention reduce the incidence of abnormal karyotype in expanding endothelial progenitor cells during passage. It is believed that enhanced karyotypic normalcy in EC. EC cells may provide a safer and/or more effective clinical outcome.

In a preferred embodiment of this aspect of the invention, a method for producing ECFCs (i.e. ECFCs®) involves passaging endothelial progenitor clones before they reach confluence. For example, cells are passaged when they reach less than about 90% confluence, or between about 70% to less than 90% confluence, or less than about 80% confluence; more preferably from about 75% to about 80% confluence prior to trypsinization and passage.

### B. Inducing Blood Vessel Formation In Vivo

Another aspect of the invention relates to inducing blood flow to a tissue in need thereof including ischemic tissue such as, but not limited to, ischemic myocardium comprising administering a mixture of ECFCs and helper cells, e.g. ASCs. To further investigate this aspect of the invention ECFCs are admixed with one or more different helper cell types and injected into NOD/SCID mice. In some experiments ECFCs® are mixed with one or more helper cell types and Matrigel™ (GFR-Matrigel:HC-Matrigel) in a 3:1 ratio of ECFCs® to helper cells and a 50:50 admixture of cells to matrix material prior to injection subcutaneously into NOD/SCID mice. Cells and matrix admixtures were harvested 14 days after injection and processed for histological examination. Human blood vessel formation was examined by H&E staining and by immunochemistry using anti-human CD31/anti-human nuclear matrix staining.

TABLE 1
Single cell type administration.

| Sample | Cells | Human Blood Vessels ^{a} | | | NuMa+ | CD31+ |
|---|---|---|---|---|---|---|
| 1 | ECFCs ® | ND | ND | ND | Yes | Yes |
| 2 | ASC | ND | ND | ND | Yes | Yes |
| 3 | WJ-MSC | ND | ND | ND | Yes | No |
| 4 | HASMC | ND | ND | ND | Yes | No |
| 5 | CD133+ | ND | ND | ND | Yes | Yes |
| | exp | | | | | |
| 6 | CD133+ | ND | ND | ND | No | No |
| | unexp | | | | | |

^{a} number human blood vessels detected per 20x field of view.
ND—none detected

As illustrated in Table no single cell type produced detectable human blood vessel formation when injected into NOD/SCID mice.

However, as shown in Table 2, when ECFCs® were mixed 3:1 with one other helper cell type, human blood vessel formation was detected in this model when the helper cells were ASCs but was not detected when ECFCs® were mixed with Whaton's jelly (WJ-MSC), human aortic smooth muscle (HASNIC), or CD133⁺ cells (expanded or unexpanded).

TABLE 2
Two-cell combinations

| Sample | Cells | Human Blood Vessels ^{a} | | | NuMa+ | CD31+ |
|---|---|---|---|---|---|---|
| 7 | ECFCs ® + | 55 | 48 | ND | Yes | Yes |
| | ASC | | | | | |
| 12 | ECFCs ® + | 67 | 36 | 76 | Yes | Yes |
| | ASC | | | | | |
| 8 | ECFCs ® + | ND | ND | ND | Yes | Yes |
| | WJ-MSC | | | | | |
| 13 | ECFCs ® + | ND | ND | ND | Yes | Yes |
| | WJ-MSC | | | | | |
| 9 | ECFCs ® + | ND | ND | ND | Yes | Yes |
| | HASMC | | | | | |
| 14 | ECFCs ® + | ND | ND | ND | Yes | Yes |
| | HASMC | | | | | |
| 10 | ECFCs ® + | ND | ND | ND | Yes | Yes |
| | CD133+ exp | | | | | |
| 15 | ECFCs ® + | ND | ND | ND | No | No |
| | CD133+ exp | | | | | |
| 11 | ECFCs ® + | ND | ND | ND | Yes | Yes |
| | CD133+ unexp | | | | | |
| 16 | ECFCs ® + | ND | ND | ND | Yes | Yes |
| | CD133+ unexp | | | | | |

^{a} number human blood vessels detected per 20x field of view.
ND—none detected

To further investigate the effect of admixing one or more helper cell types with ECFCs®, combinations of three-cell types in a ratio of 3:1:1 (ECFC:cell type 1: cell type 2) were tested. As shown in Tables 3 and 4, the combination of ECFCs®, ASCs and WJ-MSC resulted in no detectable human blood vessel growth. However all other tested 3-cell type combinations that included ECFCs® and ASCs resulted in detectable human blood vessel growth (Table 3).

TABLE 3
Three cell combinations including ECFCs ® and ASCs.

| Sample | Cells | Human Blood Vessels ^{a} | | | NuMa+ | CD31+ |
|---|---|---|---|---|---|---|
| 17 | ECFCs ® + | ND | ND | ND | Yes | Yes |
| | ASC + | | | | | |
| | WJ-MSC | | | | | |
| 21 | ECFCs ® + | ND | ND | ND | Yes | Yes |
| | ASC + | | | | | |
| | WJ-MSC | | | | | |
| 18 | ECFCs ® + | 38 | 25 | 11 | | Yes |
| | ASC + | | | | | |
| | HASMC | | | | | |
| 22 | ECFCs ® + | 16 | 10 | 13 | | Yes |
| | ASC + | | | | | |
| | HASMC | | | | | |
| 19 | ECFCs ® + | 16 | ND | 2 | | Yes |
| | ASC + | | | | | |
| | CD133+ exp | | | | | |
| 23 | ECFCs ® + | 24 | 27 | 14 | | Yes |
| | ASC + | | | | | |
| | CD133+ exp | | | | | |
| 20 | ECFCs ® + | 38 | 25 | 7 | | Yes |
| | ASC + | | | | | |
| | CD133+ unexp | | | | | |
| 24 | ECFCs ® + | ND | ND | ND | | Yes |
| | ASC + | | | | | |
| | CD133+ unexp | | | | | |

^{a} number human blood vessels detected per 20x field of view.
ND—none detected

In contrast 3-cell combinations that excluded ASCs yielded low to undetectable human blood vessel growth (Table 4.)

TABLE 4
Three-cell combinations of ECFCs ® and non-ASC helper cells.

| Sample | Cells | Human BloodVessels ^{a} | | | NuMa+ | CD31+ |
|---|---|---|---|---|---|---|
| 29 | Matrigel alone | ND | ND | ND | No | No |
| 27 | ECFCs ® + | ND | ND | ND | | Yes |
| | WJ-MSC + | | | | | |
| | CD133+ exp | | | | | |
| 32 | ECFCs ® + | ND | ND | ND | | Yes |
| | WJ-MSC + | | | | | |
| | CD133+ exp | | | | | |
| 28 | ECFCs ® + | ND | ND | ND | | Yes |
| | WJ-MSC + | | | | | |
| | CD133+ unexp | | | | | |
| 25 | ECFCs ® + | ND | ND | ND | | Yes |
| | HASMC + | | | | | |
| | CD133+ exp | | | | | |
| 26 | ECFCs ® + | 9 | 1 | ND | | Yes |
| | HASMC + | | | | | |
| | CD133+ unexp | | | | | |
| 30 | ECFCs ® + | ND | ND | ND | | Yes |
| | WJ-MSC + | | | | | |
| | HASMC | | | | | |
| 31 | ECFCs ® + | ND | ND | ND | | Yes |
| | WJ-MSC + | | | | | |
| | HASMC | | | | | |

^{a} number human blood vessels detected per 20x field of view.
ND—none detected

These results show that new blood vessel formation is enhanced in the NON/SCID mouse model when ECFCs® are administered with ASCs, alone or in combination with other helper cell types. New blood vessel formation was also detected at low levels when ECFCs® were admixed with HASMC and CD133+ (unexpanded) (Table 4).

### C. ECFCs Restore Blood Flow to Ischemic Heart

One embodiment of the present invention relates to administering an effective amount or dosage of ECFCs, preferably karyotypically normal ECFCs (e.g. ECFCs®), to restore and/of improve blood flow to ischemic tissues including, myocardial tissues.

When vasculogenesis is desirable, ECFCs are mixed with helper cells, and a suitable matrix. No evidence could be found that ASCs, mesenchymal cells of any source, or ECFCs when administered alone, or ECFCs in combination with a matrix material, induce robust vasculogenesis in comparison to ECFCs in the presence of one or more appropriate “helper” cells. Helper cells contribute to and synergize with ECFCs promoting vasculogenesis and are believed to provide a source of smooth muscle cells and/or pericytes, which are believed to stabilize nascent tube formation. Suitable helper cells include but are not limited to an appropriate source of smooth muscle cells and/or pericytes, for example, ASCs and mesenchymal endometrial cells (Cryo-Cell, Inc.). The effect of enhancing vasculogenesis by administering ECFCs in combination with helper cells is facilitated by administering the cells in a suitable matrix material. It is believed that the matrix helps concentrate the cells and prevent or reduce cell migration or dilution at a site of administration e.g. injection).

### C.1. Rat Myocardial Ischemia Model

ECFCs® are tested in a rat myocardial ischemia model to assess their ability to restore blood perfusion and improve heart function in ischemic tissue (Studies 1-3). Test animals receive ECFCs® alone, ASCs alone, ECFCs®+ASCs, or saline. Myocardial ischemia is induced in male athymic “nude” rats (Hall RHNU; Charles River Laboratories). On the day of surgery, an animal from each of four groups is prepared for cell administration by undergoing open heart surgery and ligation of the left anterior descending artery (LAD). Blood perfusion to the left ventricle is reduced sufficiently to achieve ischemia, as indicated by “blanching” of the left ventricular tissue. For Studies 1 and 2. ECFCs® are injected in a liquid carrier into the perimeter of the ischemic area in four aliquots of 20-50 ul each. Each animal receives a total of approximately 10⁶ cells/kg bodyweight. For Study 3, ECFCs® and ASCs are mixed together with a suitable matrix material (Matrigel®/Hydrogel) prior to administration to assess possible synergy in the restoration of blood flow and heart function. Endpoints are measured at 14 days (Studies 1-3) and 28 days (Study 2) post-administration by echocardiographic measurement in live animals. After sacrifice, blood vessel size and density in the ischemic tissue is evaluated by histological examination. In addition, histological samples are stained to reveal human cells in the endothelial lining of cardiac blood vessels.

### Media and Reagents

ECFCs® Growth Media. EGM™-2 (complete endothelial cell culture media containing various growth factors including VEGF).

HSC Growth Media. SLII (complete CD34⁺ cell culture media containing several growth factor cytokines and human serum albumin).

ASC Growth Media: ADSC media (complete adipose-derived stein cell media containing 10% fetal calf serum) or DMEM containing 10% fetal calf serum.

HSC Freeze Media, Dulbecco's Phosphate Buffered Saline (DPBS) containing 0.5% bovine serum albumin and 10% dimethylsulfoxide.

ECFCs® and ASCs Freeze media: Fetal calf serum containing 5% dimethylsulfoxide.

HSC Thaw Media. Dulbecco's Phosphate Buffered Saline containing 0.5% bovine serum albumin.

Extracel®™-HP kit: 50:50 mixture of Heprasil® and Gelin-S® combined with or without bFGF, VEGF and human umbilical cord extracellular matrix.

### Isolation, Expansion and Cyropreservation of ECFCs®

Cord blood mononuclear cells (MNCs) are prepared from donated umbilical cord blood by standard methods, and seeded at 5×10⁷ cells/well in collagen-coated 6-well plates containing 4 mL complete EGM®-2 media (Lonza) and placed in a humidified 37° C.; 5% (v/v) CO₂ incubator. Every 24 h for 7 days, and then every 2 days, the media is gently replaced. On day 12, clones are plucked from die wells and the colony dissociated with trypsin/EDTA. Each colony is seeded into a single well containing 2 mL complete EGM®-2 and returned to the incubator. Cells are expanded into flasks when the cells are less than about 75-80% confluent, usually between 4 to 7 days. The clonal selection and re-plating, process selects the HPP ECFCs® subpopulations of adherent cells from the MNCs in cord blood. After a third passage, expanded ECFCs® are cryopreserved in aliquots of 10⁶ cells per vial (“Passage 3” vials). Passage 3 vials are thawed and expanded to passage 4, yielding approximately 10⁸ cells, and then cryopreserved in appropriate aliquots (ECFCS®),

### Passaging, Expansion and Cryopreservation of ASCs

Cryopreserved ASCs are removed from liquid nitrogen storage and warmed in a 37° C. water bath until crystals are no longer present in the vials. The ASCs are transferred from the cryovial to 20 mL ASC complete media and plated onto a collagen coated T150. Cells are incubated overnight at 37° C. in 5% CO₂ humidified tissue culture incubator. To remove residual DMSO, tissue culture media is removed from the cells 18 hrs later, and 20 mL of fresh ASC complete media is added to the cells. ASCs are cultured until they are 85% confluent at which time the cells are passaged. Adherent ASCs are replenished with fresh ASC complete media every other day. After reaching 85% confluence, the ASCs are cryopreserved in aliquots of 10⁶ cells per vial. The cells are slowly cooled to −70° C. using a controlled rate freezer and stored in liquid nitrogen.

### Preparation of Cells for Injection

### Preparation of ECFCs® in Liquid Medium (Studies 1 & 2)

ECFCs® are removed from liquid nitrogen storage, and quickly warmed in a 37° C. water bath. Cells are transferred to pre-warmed, complete EGM™-2 and pelleted at 300-400×g for 7-10 minutes. The supernatant is removed and cells are resuspended in complete media to be cultured in suspension for 1-2 hours in a humidified 37° C.; 5% (v/v) CO₂ incubator. Cells are centrifuged at 300-400×g for 7-10 minutes, the supernatant removed, and the cells are resuspended in Dulbecco's PBS at concentrations to give a dose of 10⁶ cells/kg. The resuspended cells are drawn up in a 30G Hamilton syringe for injection.

### Preparation of EXTRACEL®/ASC/ECFCs® Admixture (Study 3)

### Cell Preparation:

Cells are cultured as described above. Adherent cells are trypsinized, centrifuged, and resuspended in EGM-2™ at 10⁷ cells/ml.

### Extracel®-HP Preparation:

Following the manufacturer's specifications (Glycosan BioSystems, Salt Lake City, Utah), one vial of Gelin-S®, one vial of Heprasil®, and one vial ExtraLink® are transferred from −20° C. to a 37° C. tissue culture incubator. The vials are pre-warmed at 37° C. for 30 minutes. Using a syringe, 1 ml of DQ water is added to each of the vials of Gelin-S® and Heprasil®. 05 ml of DQ water is added to the vial of ExtraLink®. The vials are incubated for an additional 30 minutes at 37° C. 1 ml of Gelin-S® is combined with 1 ml of Heprasil® in a 15 ml conical flask, 255 μl of Extracel-HP®+ cells are prepared per injection site.

### For Extracel-HP®:

143 μL of 50:50 mixture of Heprasil® and Gelin-S®+22 μl of PBS

For Extracel-HP®+bFGF+VEGF:

143 μL of 50:50 mixture of Heprasil® and Gelin-S®+1 μl of bFGF (100 ng/ml)+1 μl of VEGF (100 ng/ml)+20 μl of PBS

For Extracel-HP®+bFGF+VEGF+ECM:

143 μL of 50:50 mixture of Heprasil® and Gelin-S®+1 μl of bFGF (100 ng/ml)+1 μl of VEGF (100 ng/ml)+20 μl of ECM

### Per Injection Site:

30 μl of ECFCs® (at 10⁷ cells/ml)+10 μl of ASCs at 10⁷ cells/ml).

40 μl of EGM-2 (no cell control).

165 μl of appropriate Extracel-HP® mixture is added to 40 μl of cells in EGM-2 and mixed well. 50 μl of ExtraLink® is added to each sample to initiate solidification. After 15-20 minutes, the Extracel-HP® begins to solidify, and the matrix infused with cells is drawn up in a syringe for injection.

### Test and Vehicle/Control Article Administration

### Route of Administration

Test articles are administered by intramyocardial injection via a 100 μl Hamilton syringe and 28 gauge needle. The test and vehicle/control articles are administered once on Day 0, as 4 separate injections into the left ventricular free wall of the heart using a range of cell dosages and volumes. For example, approximately 1-2×10⁶ cells are administered per 100 ul volume.

About 0.3×10⁶ (1 million cells per kg) cells are provided by intramyocardial injection per infarcted animal in rats with permanent LAD ligations.

About 0.3×10⁶ cells (per heart) are provided by intramyocardial injection in rats with temporary LAD ligations. Test animals receive 4 injections each of 20-50 ul in volume.

### Animal Preparation

Unfasted nude female athymic rats (10 weeks old weighing 225-250 g) are prepared for surgery as follows:

### Pre-Operative Procedures

Anesthesia is administered and maintained as described in Table 5. Treated animals are placed on mechanical ventilation. Lactated Ringer's Solution is given subcutaneously. Routine aseptic technique is used throughout the surgery. The lateral thorax is shaved and prepared with iodine scrub, 70% isopropyl alcohol and iodine solution.

TABLE 5
Rat myocardial infarction model medications and dosages

| | INTERVAL, DOSE, AND ROUTE |
|---|---|
| DRUG | SURGERY (DAY 0) |
| Isoflurane | To effect |
| Buprenorphine | mg/kg SC (t.i.d.) on day of surgery and |
| | mg/kg SC (t.i.d.) for 3 days post-op |
| Lactated Ringer's | 3-5 ml SC |
| Solution | |

### Surgical Procedure

A midline sternotomy is performed on anesthesized animals. For non-infarcted animals, the pericardium is opened and the test article or vehicle is injected at four separate sites into the left ventricular anterior free wall of the heart. For infarcted animals, the LAD is identified and then is either permanently occluded with a suture, or is occluded temporarily for about 1 hour. Occlusion is verified by blanching of the myocardium and confirmation of characteristic changes of ischemia on the ECG waveform (e.g. ST elevation). For the one hour occlusion, the suture is removed and the infarcted area is allowed to reperfuse. Following reperfusion the test article is injected as described above and the thorax closed.

### Post-Operative Procedures

Following surgery, animals are closely monitored for physiological disturbances including cardiovascular/respiratory depression, hypothermia, and excessive bleeding from the surgical site. Long term post-operative monitoring includes inspection for signs of pain or infection. Staples, if used, are removed 7-10 days post-surgery. Any supplemental pain management and antimicrobial therapy is administered as needed.

### Echocardiographic Analysis

Animals in Study 1 are subjected to echocardiographic analysis immediately prior to surgery and again at 2 weeks post-surgery, before sacrifice. Study 2 animals are analyzed at time zero, at 2 weeks post surgery and at 4 weeks just before sacrifice.

### Antemortem Study Evaluation

### Detailed Clinical Examinations

A detailed clinical examination of each animal is performed once during each study week. Observations include evaluation of the skin, fur, eves, ears, nose, oral cavity, thorax, abdomen, external genitalia, limbs and feet, respiratory and circulatory effects, autonomic effects such as salivation, nervous system effects including tremors, convulsions, reactivity to handling, and bizarre behavior. Body weights are measured and recorded within 3 days of arrival, at least once prior to randomization, and weekly during the study.

### Postmortem Study Evaluations and Histology Preparation

At the end of the studies (Day 14 or Day 28), animals are euthanized and examined. The heart is removed and sectioned from apex to base by uniform random sampling to produce four slabs, each of about 3 mm thickness, per left ventricle. Slabs are routinely processed and paraffin embedded. Multiple histological sections of about 5 microns to 8 microns in thickness are cut from each slab and stained with hematoxylin and eosin, or picrosirius red, and immunohistochemically stained for vWF, CD31, and Human Nuclear Matrix.

### Histomorphometry:

### Assessment of Infarct Size

Sections are stained with a collagen stain (e.g., picrosirius red) to assess collagen density as a measure of infarct area. Histomorphometry is performed and the following measurements taken: 1) the inner LV circumference, 2) the outer LV circumference, 3) the outer and inner infarct arc, and 4) the area of the infarct. Data are recorded as a percent of area of LV infarcted.

### Histochemistry

### Assessment of Capillary Density:

Sections are stained with anti-rat vWF (von Willebrand's Factor). Five microscopic fields (400× magnification) from each section, cut perpendicular to the long axis of the cardiac muscle fibers, are acquired and digitized. The total number of capillaries in each field is counted using Image-Pro Plus software (Media Cybernetics, Rockville, Md.) and capillary density (capillaries/mm²) calculated,

### Assessment of Human Blood Vessel Formation:

From each block, sections are stained with anti-human CD31 to determine differentiation of transplanted progenitor cells into mature endothelial cells, and anti human nuclear matrix antibody for detection of human cells. The nuclei are counterstained with Hematoxylin

### C.2. Results (Studies 1 & 2)

When ECFCs® alone are injected in a liquid carrier directly into the myocardium in the rat myocardial ischemia model improvements in ventricular function are observed at 14 days and at day 28 after the procedure. Additionally, histological assessment of blood vessel density in control and ECFCs® ischemic tissue measured at 14 and 28 days after the procedure show significant improvement in treated animals. The mechanism of functional improvement is assessed by measuring capillary density in the infarcted areas of the control and ECFCs® treated animals. To see if any increase in capillary density is due to angiogenic and/or vasculogenic activities of ECFCs®, immunohistochemical analysis of the infarcted region is performed to evaluate whether ECFCs® had participated in vasculogenesis as evidenced by incorporation of ECFCs® into the intimae of blood vessel capillaries.

Echocardiographic evaluation of heart function is performed immediately before surgery and two weeks later, prior to sacrifice. A 2-dimensional short-axis view of the left ventricle (LV) is obtained at mid-papillary and apical levels. M-mode tracings are recorded through the anterior and posterior LV walls to allow delineation of wall thickness and motion in infarcted and non-infarcted regions of the heart. The results are recorded and LV mass determined. Relative anterior wall thickness, posterior wall thickness, and LV internal dimensions are measured, preferably from at least three consecutive cardiac cycles. Endocardial fractional shortening and midwall fractional shortening are used as indices to estimate LV systolic function.

Comparisons among groups of test animals are made using ANOVA with Tukey post-hoc comparisons to determine significance. A critical value of P<0.05 is considered a significant difference or treatment effect.

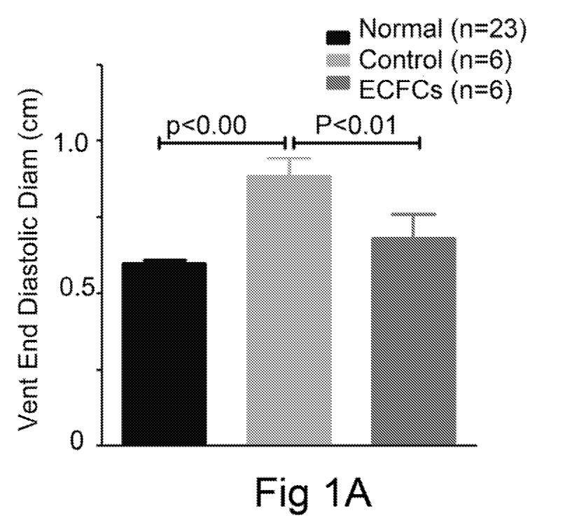

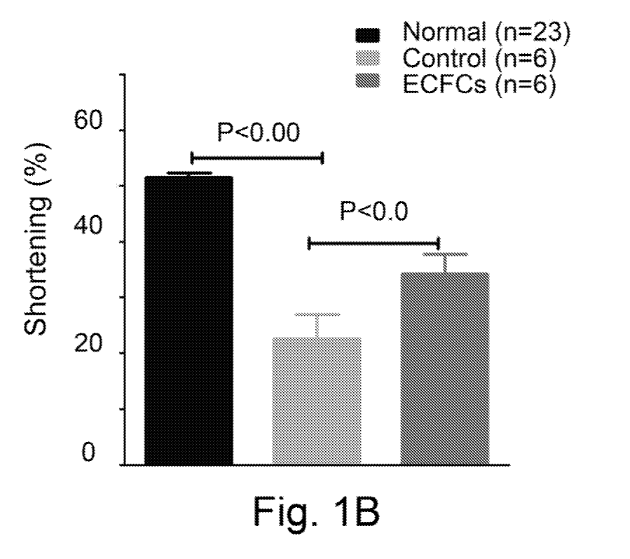

Echocardiographic measurements including left ventricular pressure and contractility in cell-treated animals are not significantly different from negative controls. In contrast, significant treatment differences are observed in left ventricular function and geometry. For example, rats induced to infarct display significantly increased left ventricular end diastolic diameter (LVEDD) after permanent artery ligation when compared to normal animals. However, following treatment with ECFCs® there is a reduction toward normal LVEDD (See FIG. 1A).

Additional treatment effects are assessed by measuring left ventricular percent shortening, sometimes referred to as fractional shortening. Percent shortening is significantly reduced after coronary artery ligation followed by control saline injections. However, rats treated with ECFCs® show significant improvement compared to saline controls (FIG. 1B). As another test of the effectiveness of the treatment, measurements are also taken of anterior wall thickness. Significant reduction in anterior wall thickness occurs in rats that undergo coronary artery ligation followed by saline injections. However, rats treated with ECFCs® show significant increase in anterior wall thickness. In contrast, no differences are observed in the posterior wall thickness in any of the treated groups compared to controls (data not shown).

Ejection fraction is an additional independent measure of heart function, routinely estimated in short-axis mode from digital images at the mid-papillary level of the heart. Because many of the infarcts induced by the ligation procedure are at the lower apical portion of the heart, ejection fraction measurements taken at mid-papillary level are not expected to show dysfunction. Thus, ejection fraction is determined at the apical portion of the heart. Animals treated with ECFCs® show an improvement in ejection fraction (data not shown).

In summary, direct injection of ECFCs® in a liquid carrier is an effective means to improve cardiac function associated with ischemic injury in a rat model.

The efficacy of this aspect of the invention is further demonstrated in histological sections taken from hearts of animals treated as described herein. The data demonstrate that the ECFCs® group shows a greater trend to reduction in infarct size compared to control. A histological section taken at level 2, cranial to the apex, from the heart of a rat subjected to permanent left anterior descending coronary artery ligation and intramyocardial injection of saline solution (sham control) two weeks previously shows morphology (Picrosirius red stain; 20× instrument magnification) typical of the model with substantial anterior left ventricular free wall infarction and marked thinning and fibrosis (red staining highlights fibrillar collagen) of the affected region. A histological section taken from level 2, cranial to the apex, from the heart of a rat given similar coronary ligation but infected with ECFCs® at the time of coronary ligation shows greatly reduced myocardial wall thinning with substantial viable, contractile tissue remaining in the area at risk in the ECFCs® treated animal.

Significant functional improvements in both the left ventricular end diastolic diameter, as well as the percent left ventricular short-axis diameter shortening are also measured at day 28 in Study 2.

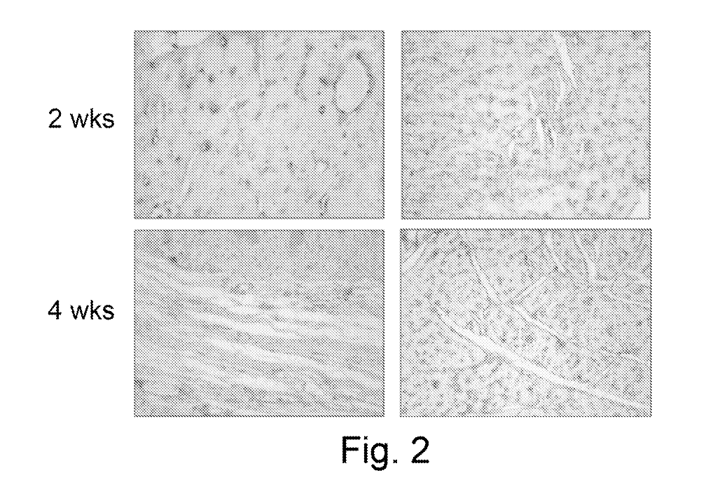

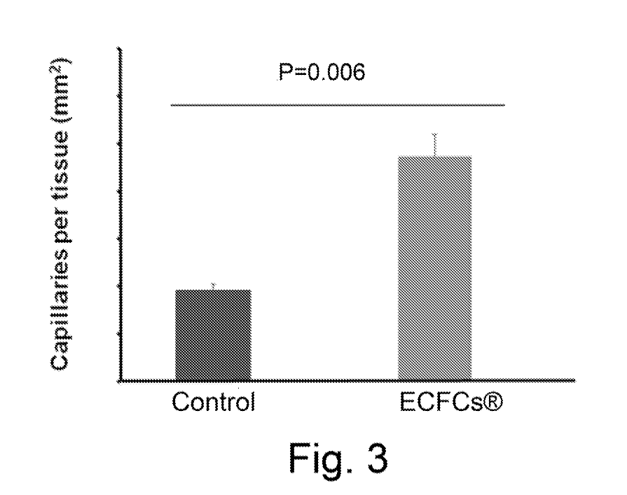

Capillary density measurements provide a basis to assess the effect of treatment on angiogenesis, which is one possible mechanism for therapeutic benefit. At day 14 there is no significant effect of treatment on capillary density (P=0.553) (FIG. 2; left panels, top and bottom). However, at day 28 significant increase in capillary density is observed (FIG. 2; right panels, top and bottom). This is expressed quantitatively in FIG. 3. (Control=48±3 capillaries/mm²; ECFCs®=119±12 capillaries/mm²). Histological examination of infarcted rat tissues in the zones where capillary density had been augmented by ECFCs® treatment using a human endothelium specific anti-CD31 antibody reveals that no human cells could be detected in the intimae of the capillaries. This indicates that injection of ECFCs® into ischemic rat heart induces an increase in capillary density most likely as a paracrine induction of angiogenesis,

Injection of ECFC/Helper Cell/Matrix Admixture into Myocardium (Study 3)

ECFCs have been shown to undergo vasculogenesis in a subcutaneous collagen/fibronectin implant in the NOD/Scid immunodeficient mouse, with concomitant inosculation as evidenced by blood vessels in the implant that contain and circulate host blood. Endothelial cells have been shown to form vascular networks in (Yoder et al., Blood, 109, 1801-1809, 2007) and in healthy tissue (Malero-Martin et al Circulation Res, (2008); 103 194-202). However, to date there has not been any demonstration that de-novo vasculogenesis can occur when a cell mixture in an injectable matrix is administered directly into a pathologically ischemic tissue.

Vasculogenesis, the process of inducing new blood vessel formation de novo, is believed to be necessary or at least desirable in many instances of ischemia including myocardial ischemia as a means to increase the likelihood of stopping and/or reversing ischemic tissue damage, and in facilitating remodeling of the tissue to regain normal function. Preferably the admixture of cells for this aspect of the invention further comprises a non-toxic extracellular matrix. In a preferred embodiment. ECFCs are karyotypically normal (e.g. ECFCs®). For example, in one embodiment ECFCs® and ASCs (human adipose tissue stromal cells) are admixed in an extracellular matrix, for example Matrigel® or Extracel® prior to administration, for example, by injection into an ischemic tissue including an ischemic region of myocardium. Injections of an ECFC®/ASC/matrix composition into ischemic rat myocardium results in the formation of stable human blood vessels within the ischemic area of the rat heart demonstrating that vasculogenesis occurs within the ischemic myocardial environment

### Results

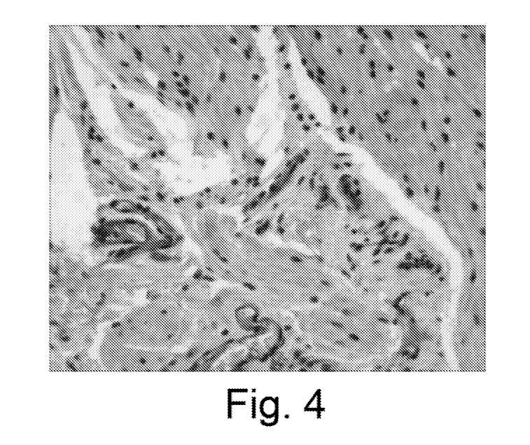

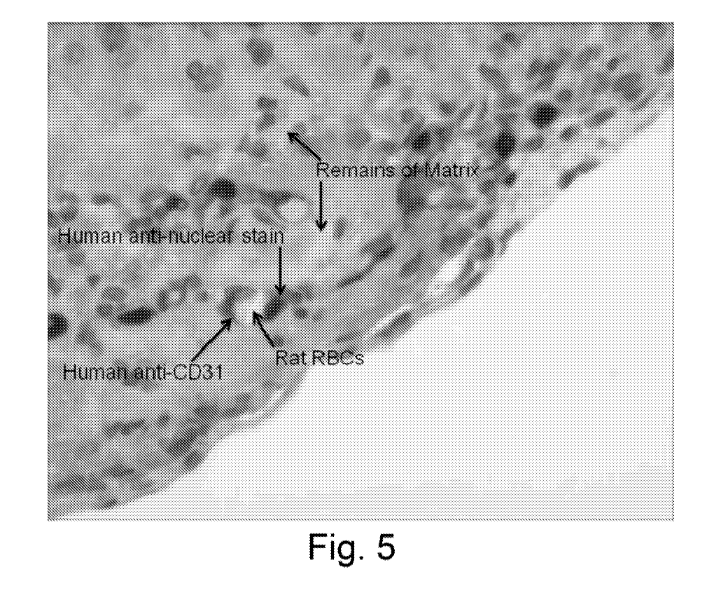

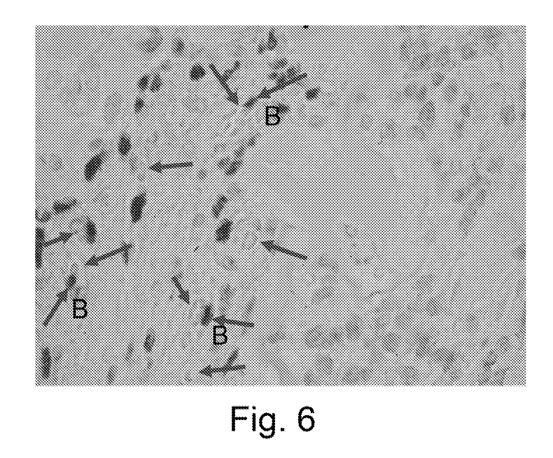

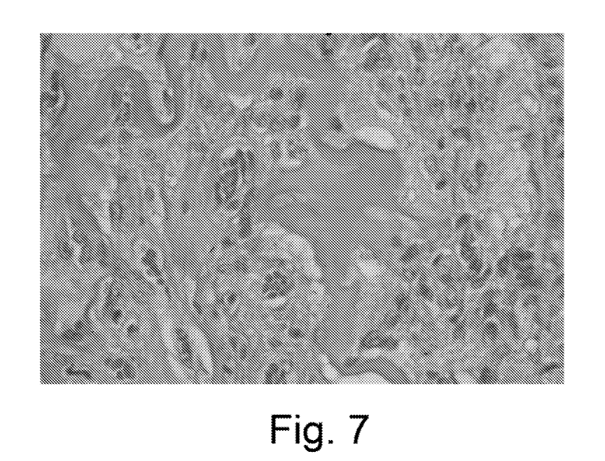

A control histological section of healthy human colon, showing CD31 staining (human anti-CD31 bound to a red dye) and nuclei staining (human anti-nuclear matrix protein bound to a brown dye) is shown in FIG. 4. A histological section of normal rat myocardium injected with a mixture of ECFCs®, ASCs, and Extracel® showing human anti-CD31 and human nuclei in vessels is shown in FIG. 5. The vessels contain rat red blood cells, demonstrating that the human vessels have inosculated with the rat blood supply. FIG. 6 shows the presence of human blood vessels in the ischemic rat myocardium injected with a mixture of ECFCs®, ASCs, and Extracel®, thereby establishing that human endothelial cells line the blood vessels. The intimal areas of the blood vessels are identified by unlabeled arrows in FIG. 6, showing the line that divides the lumen of the blood vessel from the surrounding tissues. Arrows labeled “B” in FIG. 6 identify human nuclei in cells that are integrated into the Marital lining of the blood vessel—that is endothelial cells of human origin and therefore derived from ECFCs®. Other human nuclei are present which are not associated with blood vessels. In FIG. 7, the H&E staining of an adjacent section shown in FIG. 6 clearly shows rat red blood cells only in the area of the ischemic heart that contained the human blood vessels. There is no evidence of red blood cells, or vessel like structures, in areas adjacent to the Extracel® matrix where no human cells are observed.

Thus, one aspect of the invention relates to promoting an angiogenic effect by administering ECFCs alone, preferably in a liquid carrier, in another aspect, the invention relates to inducing a vasculogenic effect, by administering or co-administering ECFCs in admixture with one or more helper cell types, preferably in a suitable matrix material. Preferably, the ECFCs have a normal karyotype, most preferably ECFCs are ECFCs.

This aspect of the invention is expected to provide a variety of therapeutic options to maximize clinical benefit according to the need of each patient. Different physiological effects can be induced by administering ECFCs alone or in combination with helper cells. Treatments can be targeted at increasing angiogenesis alone or in promoting both angiogenesis and vasculogenesis. For example, a paracrine angiogenic effect can be induced by administering ECFCs® alone directly into injured tissue surrounding an infarct zone. Alternatively, angiogenic and vasculogenic effects can be induced by co-administering ECFCs® and one or more helper cell types, e.g. ASCs in a relevant non-toxic matrix. Inducing vasculogenesis and/or angiogenesis is expected to lead to more effective treatments through restoration of blood flow, accelerated tissue remodeling, and recovery of function in ischemic tissues.

### D. Clinical Applications

The methods of the present invention provide improved therapeutic methods for treating diseases associated with reduced blood flow and/or insufficient perfusion, for example, myocardial infarction (MI), congestive heart failure (CHF), stroke, peripheral artery disease (PAD), and ischemic disorders including myocardial ischemia generally and more specifically acute myocardial ischemia, chronic myocardial ischemia, myocardial infarction. CAD, left ventricular dysfunction, and end-stage ischemic heart disease. Suitable patients include but are not limited to those with severe ischemic heart failure and chronic coronary artery disease.

The present invention relates in part to the use of ECFCs, alone or in combination with helper cells and/or other agents such as a suitable matrix material and growth factors, for therapeutic purposes. In a preferred embodiment ECFCs are karyotypically normal (e.g. ECFCs®) that are administered as an admixture with helper cells, preferably ASCs and a matrix material. It is anticipated that use of karyotypically normal ECFCs in clinical applications will result in enhanced efficacy and/or safety. In one embodiment of this aspect of the invention, ECFCs alone or in combination with one or more helper cell types are administered to a patient in need thereof to prevent or treat diseases that include, for example, myocardial infarction, congestive heart failure, stroke, peripheral artery disease, and ischemic disease, including limb ischemia or myocardial ischemia including acute and chronic myocardial ischemia. In a preferred embodiment, ECFCs® are administered as an admixture with ASCs and matrix material to a site at or near an ischemic region of tissue. For example, ECFCs may be admixed with adipose stromal cells and a matrix material, e.g. Extracel®, for purposes of administration to an ischemic region of the heart. The composition and method may also further include one or more growth factors including but not limited to VEGF, bFGF, PDGF-1, PDGF-2, IGF, FGF-1, FGF-2. Preferably ECFCs are combined with ASCs and the combination mixed with a suitable matrix material, e.g. Extracel-HP®, Gelfoam® (Pharmacia & Upjohn), or a collagen/fibronectin mixture. ECFCs and ASCs may be combined in as ratio of 10:1 to 0.5:1, respectively; alternatively in a ratio of 5:1 to 1:1; preferably in a ratio of 3:1 to 2:1.

In another aspect of the invention, a cell-based composition comprising ECFCs; or ECFCs and helper cells; or ECFCs, helper cells, and a matrix material are used in conjunction with other treatment options known to the skilled artisan including the use of stems or other vascular prosthetic devices used in treating ischemia disorders including myocardial ischemia. ECFC compositions contemplated in this aspect of the invention may be administered before, during, or after a procedure that places such a device(s).

The methods of the invention are expected to induce blood vessel formation and improve blood flow thereby providing clinical benefit to patients in need thereof. The present invention is expected to reduce the risk of and/or prevent myocardial infarction and/or reduce post-infarction damage to heart muscle, for example scarring and fibrosis. The methods are also expected to reduce the risk amid/or damage from ischemia, stroke, congestive heart failure and peripheral artery disease by inducing vessel formation and increasing blood, flow to a tissue(s) and/or organ(s) in need thereof.

In another embodiment of the invention, a patient in need receives an allogeneic transfer of ECFCs, alone or in combination with one or more other agents including helper cells, and/or growth factors and/or matrix materials, either systemically or by direct injection, into an ischemic area of tissue, for example, an ischemic area of the heart. For illustrative purposes and without intending to limit the scope of the invention, the method is described hereinbelow with reference to myocardial ischemia though it should be understood that other types of ischemia are also expected to be amenable to treatment with the products and methods of the present invention, it should further be understood that in the preferred embodiment, ECFCs are karyotypically normal; in the most preferred embodiment ECFCs are ECFCs®.

ECFCs can be delivered into the ischemic myocardium either by invasive or noninvasive means. For example, ECFCs can be administered by systemic infusion or by local transplantation at an ischemic site in the myocardium or region surrounding an ischemic site. In other aspects of the invention, ECFCs are administered by injection transendocardially or trans-epicardially, allowing the cells to penetrate the protective surrounding membrane. A preferred embodiment of the invention includes use of a catheter-based delivery of ECFCs for transendocardial injection. The use of a catheter provides a less invasive method of delivery, avoiding the need to open the chest cavity and provides for quicker recovery. In a preferred embodiment, cells are injected through a cardiac catheter into the wall of regions of the heart that are ischemic. Cells may also be injected into healthy surrounding tissue regions. Preferably, treatment comprises one or more injections of cells to a plurality of sites at or near a site of ischemic injury. Invasive means include, but are not limited to, epicardial injection of cells into a surgically exposed heart, directly into an ischemic area, an infarcted area, or into viable myocardial tissue surrounding diseased areas.

Other preferred means for delivering ECFCs to damaged myocardium include catheter-based transendocardial injection which provides the benefit of less invasiveness and the ability to visually map the heart and determine the best place to inject cells. For example, hibernating myocardium may be a preferred target for this type of procedure.

An appropriate dosage of ECFCs to administer or deliver to a patient according to the present invention will depend on the particular patient, the condition being treated and will involve such factors as mode of administration, patient bodyweight, and severity of the disease or condition being treated. Generally, an effective dosage would fall within a range of about 1×10⁵ to about 1×10⁷ ECFCs per kg bodyweight; or about 1×10⁶ to about 1×10⁷ cells per injection site. When ECFCs are co-administered with helper cells, the ratio of ECFCs to helper cells is about 1:1 to about 20:1 preferably about 2:1 to about 3:1. In one embodiment, when the helper cells are ASCs the ratio could also be about 6:4 to about 1:9. In certain embodiments, a therapeutically effective dose of ECFCs cells is applied, delivered, or administered to the heart or implanted into the heart of a patient in need thereof. An effective dosage is an amount or number sufficient to achieve a beneficial or desired clinical result. An effective dose can be administered in one or more administrations. However, the precise determination of an effective dose will be based on factors individual to each patient, including bodyweight, age, size of the infarcted area, and amount of time elapsed since occurrence of the damage. The treating physician, surgeon, or cardiologist, would be able to determine the number of cells which would constitute an effective dose without being subject to undue experimentation, from this disclosure and the knowledge in the art.

In another aspect of the invention, ECFCs are delivered to the heart, specifically to the border area of the infarct. As one skilled in the art would be aware, the infarcted area is generally visible to the naked eye, allowing targeted placement of stern cells to the infarcted area.

The present invention also contemplates methods and kits in which ECFCs, preferably karyotypically normal ECFCs such as ECFCs®, are combined with or co-administered with one or more other agents including, for example, CD34⁺ cells β-blockers, diuretics. Ca-channel blockers, ACE inhibitors, proteins or peptides and growth factors. In one embodiment, co-administration involves administering ECFCs, sequentially, concurrently, or simultaneously with one or more other agents. Another embodiment relates to a kit which includes a container with ECFCs, optionally also including one or more containers with other agents selected from the group consisting of helper cells, matrix material, CD34⁺ cells, β-blockers, diuretics, Ca-channel blockers, ACE inhibitors, proteins or peptides, and growth factors. A kit according to the present invention may also include devices such as a stent(s), catheter, or syringe. Another embodiment relates to the ability to directly covalently attach proteins, peptides growth factors, cytokines and antibiotics via a reactive thiol contained within a modified version of Extracel®, Heprasil® a combination of thiol-modified hyaluronan, HA, and thiol-modified heparin). Gelin-S®(thiol-modified gelatin), and Extralink® (a thiol-reactive crosslinker, polyethylene glycol diacrylate, PEGDA).

The invention has been described with reference to various illustrative embodiments and techniques. However, it should be understood that many variations and modifications as are known in the art may be made while remaining within the scope of the claimed invention. The examples that follow are illustrative and are not intended to be limiting.

### Example 1

### Preparation of ECFCs from Cord Blood

The mononuclear cell fraction (MNCs) from human cord blood was isolated by the standard ficoll-paque density gradient centrifugation technique. MNCs are plated at 50×10⁶ cells/well in collagen-coated 6-well plates with 4 ml warm EGM-2 per well. On day 1, wells are washed slowly with 2 ml EGM-2 (EBM-2 media+bullet kit+10% FBS+1% Pen/Strep) and replaced with 4 ml EGM-2. Media is changed daily for 7 days (4 ml warm EGM-2 with care to prevent washing off adherent ECFCs). After 7 days, media is changed three times per week, for example every Monday, Wednesday, Friday. Colonies may become visible around day 4. ECFCs colonies generally start to appear between day 4 to day 12. When colonies are about 0.5 cm in diameter or larger, the are plucked. Clones are passaged to larger containers when they reach about 75-80% confluence. When P3 clones are ready to be passaged they are frozen and quality control testing (QC) is performed on each done including Matrigel® tube formation, cell surface markers, and expansion. Clones that pass the QC tests are expanded to P4 via T150 flasks or roller bottles and thereafter frozen in liquid nitrogen. Each batch of frozen P4 cells is tested for mycoplasma and bacterial contamination and is karyotyped. ECFCs are cryopreserved in vapor phase liquid nitrogen.

### Example 2

### ECFCs Administered to Myocardial Ischemia Patient

A 60 year old male patient presents with chest pain induced by moderate exercise (angina) having a blood pressure of 160/90, a heart rate of 90 and a bodyweight of 210 lb. Clinical examination including ETT (exercise treadmill testing) and AECG (ambulatory electrocardiogram) as well as serum lipid profiling (showing elevated LDL) leads to a diagnosis of CAD with myocardial ischemia. The patient is placed on 81 mg daily aspirin, a beta-blocker, a fat-restrictive diet and an aggressive treatment with a statin drug (Lipitor 80 mg/d) and 2 gm fish oil daily to lower his elevated triglycerides and LDL cholesterol levels. After 6 months treatment the patient still experiences angina with moderate level activity. The patient is scheduled for intracardial administration of an ECFC-based composition comprising a mixture of ECFCs®. ASCs and a suitable matrix material in a 2:1 ratio of ECFCs®:ASCs at a concentration of about 1×10⁷ cells/ml. The cells are mixed with the matrix immediately prior to injection. Cells are administered to the patient using a cardiac catheter. About 100 μl of the cell-matrix mixture is injected at each of four roughly equivalently distributed places in the ischemic region, providing about 1×10⁶ cells per site of injection. After 2 days in the hospital the patient is released. Post-operative MSCT scans at 2 and 6 months show significantly improved cardiac blood flow and the patient reports that exercise no longer induces angina.

1. A method for treating a patient suffering from an ischemia-related disease comprising administration of an effective dose of a composition selected from the group consisting of ECFCs and an admixture of ECFCs, helper cells and a matrix material.

2. A method as in claim 1 wherein said ischemia-related disease is selected form the group consisting of myocardial ischemia, coronary artery disease, peripheral artery disease, myocardial infarction, stroke, congestive heart failure, wound healing and critical limb ischemia.

3. A method as in claim 2 wherein said administration is by injection or surgical implantation of said composition at a site of ischemic injury.

4. A method as in claim 2 wherein said helper cells are ASCs and said ECFCs are karyotypically normal.

5. A method as in claim 4 wherein said method improves blood flow at a site of ischemic tissue damage in said patient.

6. A method as in claim 5 wherein said ischemic tissue damage occurs in the heart of said patient.

7. A method as in claim 6 wherein said ECFCs are administered by intramyocardial injection or surgical implantation.

8. A method as in claim 7 wherein said disease is myocardial ischemia.

9. The method of claim 8 wherein said administering comprises the steps of a) injecting or surgically implanting ECFCs® in a peri-infarct zone and b) injecting or surgically implanting a mixture of ECFCs®, helper cells and a matrix material in the infarct zone.

10. The method of claim 8 wherein said composition is administered by intramyocardial injection at or near a site of ischemia.

11. (canceled)

12. A pharmaceutical composition for enhancing blood flow to a mammalian tissue comprising karyotypically normal ECFCs, helper cells and matrix material.

13. A pharmaceutical composition as in claim 12 wherein said ECFCs are ECFCs® and said helper cells are ASCs.

14. A pharmaceutical composition as in claim 12 wherein said helper cells comprise two different cell types wherein a first cell type is ASCs and a second cell type is selected from the group consisting of mesenchymal endometrial cells, HASMC and CD133+.

15. Use of the composition of claim 14 in the preparation of a medicament for the repair of tissue damaged by ischemic injury.

16. (canceled)

17. A process for preparing a population of ECFCs enriched for karyotypically normal cells comprising:
a. isolating the mononuclear cell fraction (MNCs) from a suitable human source;
b. plating MNCs at about 50×10⁶ cells/well;
c. after about 4 days to about 28 days isolating individual ECFCs clonal colonies;
d. passaging isolated clones when they reach less than about 90% confluence, and
e. verifying that the cells from said isolated clones have a substantially normal karyotype.

18. A population of substantially enriched, karyotypically normal, ECFCs produced by the process of claim 17.

19. (canceled)

20. A pharmaceutical kit for therapeutic use comprising a vessel containing ECFCs produced by the process of claim 17 and optionally containing one or more additional vessels containing an agent selected from the group consisting of helper cells, pharmaceutical excipients, and growth factors.
